(19) United States
(12) Patent Application Publication
(10) Pub. No.: US 2022/0289843 A1
(43) Pub. Date: Sep. 15, 2022

(54) ANTI-CD19 ANTIBODIES AND USES THEREOF

(71) Applicant: Elpis Biopharmaceuticals, Lexington, MA (US)

(72) Inventors: Yan CHEN, Lexington, MA (US); Jenna NGUYEN, Lexington, MA (US); Kehao ZHAO, Lexington, MA (US)

(21) Appl. No.: 17/636,699

(22) PCT Filed: Aug. 19, 2020
(86) PCT No.: PCT/US2020/047035

§ 371 Date: Feb. 18, 2022

Related U.S. Application Data
(60) Provisional application No. 62/888,724, filed on Aug. 19, 2019.

(51) Int. Cl. C07K 16/28

(52) U.S. Cl. CPC C07K 16/2803; C07K 16/2809; C07K 2317/565; C07K 2317/31; C07K 2317/622; C07K 2317/76; C07K 2317/92; C07K 2317/24

(57) ABSTRACT

Disclosed herein are high affinity anti-CD19 antibodies and methods of using such for therapeutic and/or diagnostic purposes. Also provided herein are methods for producing such anti-CD19 antibodies. The anti-CD19 antibodies disclosed herein showed high stability as determined by thermal shift assays and bind different CD19 epitopes as FMC63. The anti-CD19 antibody clone used for developing immunotherapeutic agents such as tisagenlecleucel.

### CROSS-REFERENCE TO RELATED APPLICATIONS

This application claims the benefit of priority to U.S. Provisional Patent Application No. 62/888,724, filed Aug. 19, 2019, which is hereby incorporated by reference in its entirety.

### BACKGROUND OF THE INVENTION

B-lymphocyte antigen CD19 is a member of the immunoglobulin super family expressed primarily on B lineage cells and follicular dendritic cells. It has been reported that CD19 acts as an adaptor protein to recruit cytoplasmic signaling proteins and as a modulator (via the CD19/CD21 complex) to decrease the threshold for the signaling pathway meditated by B cell receptors.

CD19 has been established as a promising biomarker for B lymphocyte development and lymphoma diagnosis. It is also a promising target for leukemia immunotherapies.

### SUMMARY OF THE INVENTION

The present disclosure is based, at least in part, on the development of superior anti-CD19 antibodies having high binding affinity and specificity to CD19 expressed on cell surface. The anti-CD19 antibodies disclosed herein showed high stability as determined by thermal shift assays and bind different CD19 epitopes as FMC63, the anti-CD19 antibody clone used for developing immunotherapeutic agents such as tisagenlecleucel.

Accordingly, the present disclosure provides, in some aspect, an isolated antibody that binds CD19, wherein the antibody binds to the same epitope as a reference antibody or competes against the reference antibody from binding to CD19. The reference antibody can be EP142-D9, EP187-A12, EP188-A01, or EP188-B10. In some specific examples, the reference antibody can be EP187-A12. In other examples, the reference antibody can be EP-188A01. In yet other examples, the reference antibody can be EP188-B10. Such anti-CD19 antibodies may have a binding affinity of less than 10 nM (e.g., less than 1 nM) to CD19 expressed on cell surface.

In some embodiments the anti-CD19 antibody may comprise: (a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or (b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

In some embodiments, the anti-CD19 antibody disclosed herein may collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

In some embodiments, the anti-CD19 antibody disclosed herein may comprise a V_{H} that is at least 85% identical to the V_{H} of the reference antibody, and/or a V_{L} that is at least 85% identical to the V_{L} of the reference antibody. In some examples, the anti-CD19 antibody may comprise the same heavy chain complementary determining regions (HC CDRs) and the same light chain complementary determining regions (LC CDRs) as the reference antibody. In specific examples, the anti-CD19 antibody may comprise the same V_{H} and the same V_{L} as the reference antibody.

Any of the anti-CD19 antibodies disclosed herein can be a human antibody or a humanized antibody. The antibody may be a full-length antibody or an antigen-binding fragment thereof. Alternatively, the antibody may be a single-chain antibody (scFv). Examples include SEQ ID NOs:11-14.

In some embodiments, the present disclosure provides bispecific antibodies that bind CD19 and a second antigen. For example, the second antigen can be CD3. In some embodiments, the bispecific antibody may comprise a first scFv that binds CD19 and a second scFv that binds CD3, for example, those set forth in the present disclosure. For example, the first anti-CD19 scFv may be derived from any of the exemplary anti-CD19 antibodies disclosed herein (e.g., having the same heavy chain and light chain CDRs or having the same VH and VL chains as the exemplary antibody). In some instances, the first scFv antibody may comprise the amino acid sequence of any one of SEQ ID NOs: 11-14. In some examples, the second scFv (e.g., specific to CD3) may comprise a heavy chain variable domain comprising the amino acid sequence of SEQ ID NO: 42 and a light chain variable domain comprising the amino acid sequence of SEQ ID NO: 43. In specific examples, the bispecific antibodies disclosed herein may comprise the amino acid sequence of any one of SEQ ID NOs: 40, 45, 47, and 49. Such a bispecific antibody may further comprise an N-terminus signal peptide (e.g., SEQ ID NO: 41).

In another aspect, the present disclosure provides a nucleic acid or a set of nucleic acids, which collectively encodes any of the anti-CD19 antibodies disclosed herein. In some embodiments, the nucleic acid or the set of nucleic acids can be a vector or a set of vectors, for example, expression vectors. Also provided herein are host cells comprising any of the nucleic acids or the sets of nucleic acids disclosed herein, as well as pharmaceutical compositions comprising any of the anti-CD19 antibodies disclosed herein, any of the encoding nucleic acids or sets of nucleic acids, or host cells comprising such, and a pharmaceutically acceptable carrier.

In yet another aspect, the present disclosure provides a method for inhibiting CD19 in a subject, comprising administering to a subject in need thereof any effective amount of any of the anti-CD19 antibodies disclosed herein, the encoding nucleic acids, or the pharmaceutical composition comprising such. The subject may be a human patient having CD19⁺ pathogenic cells, for example, CD19⁺ cancer cells. In some examples, the subject is a human patient having a cancer (e.g., a hematopoietic cancer). Also within the scope of the present disclosure are pharmaceutical compositions as disclosed herein for use in treating a disease comprising CD19⁺ pathologic cells such as those described herein, as well as use of any of the anti-CD19 antibodies disclosed herein for manufacturing a medicament for use in treating any of the target diseases as also disclosed herein.

Further, the present disclosure provides a method for detecting presence of CD19 (e.g., CD19 expressed on cell surface), comprising: (i) contacting an antibody of any one of claims 1-12 with a sample suspected of containing CD19, and (ii) detecting binding of the antibody to CD19. The antibody may be conjugated to a detectable label. In some embodiments, the contacting step may be performed by administering the antibody to a subject.

The present disclosure also provides a method of producing an antibody binding to CD19, comprising: (i) culturing the host cell disclosed herein under conditions allowing for expression of the antibody that binds CD19; and (ii) harvesting the antibody thus produced from the cell culture.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is an illustrative diagram showing an exemplary strategy for enriching high affinity CD19 binders from antibody libraries such as scFv libraries and single heavy chain (V_{H}) libraries.

FIG. 2A and FIG. 2B are diagrams showing exemplary single-chain (scFv) CD19 binders (FIG. 2A) and exemplary single heavy chain variable domain (V_{H}) CD19 binders (FIG. 2B) obtained from scFv and V_{H} libraries via four rounds of mRNA display selections followed by ELISA screening of individual positive clones.

FIG. 3 is a diagram showing binding activity of exemplary antibodies to HEK293 cells expressing surface CD19.

FIG. 4 is a diagram showing titration curve of the indicated exemplary scFv anti-CD19 antibodies for binding to CD19-expressing HEK293 cells.

FIGS. 5A-5D include charts showing titration curves of exemplary scFv anti-CD19 antibodies for binding to CD19-expressing HEK293 cells in the presence or absence of FMC63. FIG. 5A: EP187-A12; FIG. 5B: EP188-A01; FIG. 5C: EP188-B10; and FIG. 5D: EP142-D09.

FIG. 6 is a photo showing immunohistochemistry (IHC) staining of endogenous CD19-positive cells using exemplary anti-CD19 scFv EP187-A12.

FIG. 7 is a chart showing anti-CD19 antibody binding activity to cells expressing recombinant or endogenous CD19. For each tested anti-CD19 scFv antibody tested, bars from left to right correspond to K562 cells, CD19 K562 cells, CD19 HEK293 cells, Daudi cells, and Raji cells.

FIG. 8A and FIG. 8B are diagrams showing bispecific antibody binding activity to CD3+ Jurkat cell as measured by FACS (FIG. 8A) and ELISA (FIG. 8B).

FIG. 9A and FIG. 9B are diagrams showing cytotoxicity activity of BiTE antibodies as determined by a CTL assay (FIG. 9A) and an ELISA assay measuring cytokine secretion (FIG. 9B).

### DETAILED DESCRIPTION OF THE INVENTION

Provided herein are antibodies capable of binding to human CD19 (“anti-CD19 antibodies”), particularly CD19 expressed on cell surface. The anti-CD19 antibodies disclosed herein show high binding affinity to CD19 (e.g., cell-surface CD19), high stability, and/or bind to different CD19 epitopes as FMC63, a murine anti-CD19 antibody used in various therapeutic agents targeting CD19. Further, when comprising a moiety for engaging immune cells (e.g., a binding moiety to T cells), the exemplary anti-CD19 antibodies showed strong cytotoxicity against CD19⁺ cancer cells, indicating that the anti-CD19 antibodies disclosed herein are expected to show anti-cancer effects, particularly against cancers involving CD19⁺ cancer cells.

CD19 is a 95 kDa transmembrane glycoprotein expressed primarily on B lineage cells and follicular dendritic cells. It is a member of the immunoglobulin super family CD19 molecules from various species are well known in the art. For example, the amino acid sequence of human CD19 can be found under GenBank accession no. AAA69966.

CD19 plays essential roles in B cell malignancies and autoimmunity. For example, CD19 is reported to be expressed on the surface of cancer cells in 90% of acute lymphoblastic leukemia (ALL) patients, as well as on cancer cells of B-cell non-Hodgkin's lymphoma (NHL) and chronic lymphocytic leukemia (CLL) patients. Therefore, CD19 has been considered as a promising target for immunotherapy of cancers of B cell lineage. Stanciu-Herrera et al., Leuk Res. 2008; 32:625-32; and Le Gall et al., FEBS Lett. 1999; 453:164-8.

Thus, the anti-CD19 antibodies disclosed herein can serve as therapeutic agents for treating diseases associated with CD19, for example, cancers of B-cell linage. In addition, the anti-CD19 antibodies disclosed herein can serve as diagnostic agents for detecting presence of CD19, e.g., CD19-positive cells. The antibodies disclosed herein may also be used for research purposes.

### I. Antibodies Binding to CD19

The present disclosure provides antibodies binding to CD19, for example, human CD19. In some embodiments, the anti-CD19 antibodies disclosed herein are capable of binding to CD19 expressed on cell surface. As such, the antibodies disclosed herein may be used for either therapeutic or diagnostic purposes to target CD19-positive cells (e.g., leukemia cells). As used herein, the term “anti-CD19 antibody” refers to any antibody capable of binding to a CD19 polypeptide (e.g., a CD19 polypeptide expressed on cell surface), which can be of a suitable source, for example, human or a non-human mammal (e.g., mouse, rat, rabbit, primate such as monkey, etc.).

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-CD19 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-CD19 antibody described herein may be a full-length antibody, which contains two heavy chains and two light chains, each including a variable domain and a constant domain. Alternatively, the anti-CD19 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-CD19 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-CD19 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-CD19 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues.

Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-CD19 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-CD19 antibodies described herein specifically bind to the corresponding target antigen (e.g., CD19) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (CD19) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method). In some examples, the anti-CD19 antibody disclosed herein does not bind to the same epitope as FMC63. In other examples, the anti-CD19 antibody binds to a CD19 epitope that is not overlapping with the CD19 epitope to which FMC63 binds. The V_{H} and V_{L} sequences of FMC63 are well known in the art and provided in Table 1 below:

TABLE 1
Amino Acid Sequences of FMC63

| SEQ | | |
|---|---|---|
| ID | Descrip- | |
| NO: | tion | Sequence |
| 1 | FMC63 | EVKLQESGPGLVAPSQSLS |
| | V_{H} | VTCTVSGVSLPDYGVSWIR |
| | | QPPRKGLEWLGVIWGSETT |
| | | YYNSALKSRLTIIKDNSKS |
| | | QVFLKMNSLQTDDTAIYYC |
| | | AKHYYYGGSYAMDYWGQGT |
| | | SVTVSS |
| 2 | FMC63 | DIQMTQTTSSLSASLGDRV |
| | V_{L} | TISCRASQDISKYLNWYQQ |
| | | KPDGTVKLLIYHTSRLHSG |
| | | VPSRFSGSGSGTDYSLTIS |
| | | NLEQEDIATYFCQQGNTLP |
| | | YTFGGGTKLEIT |

In some embodiments, an anti-CD19 antibody as described herein has a suitable binding affinity for the target antigen (e.g., CD19) or antigenic epitopes thereof. As used herein, “binding affinity” refers to the apparent association constant or K_{A}. The K_{A} is the reciprocal of the dissociation constant (K_{D}). The anti-CD19 antibody described herein may have a binding affinity (K_{D}) of at least 100 nM, 10 nM, 1 nM, 0.1 nM, or lower for CD19. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-CD19 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-CD19 antibody disclosed herein has an EC₅₀ value of lower than 10 nM, e.g., <1 nM, <0.5 nM, or lower than 0.1 nM, for binding to CD19-positive cells. As used herein, EC₅₀ values refer to the minimum concentration of an antibody required to bind to 50% of the cells in a CD19-positive cell population. EC₅₀ values can be determined using conventional assays and/or assays disclosed herein. See, e.g., Examples below.

A number of exemplary anti-CD19 antibodies are described in the present disclosure and provided by amino acid sequence as below, namely antibodies: EP187-A12; EP188-B10; EP142-D09; and EP188-A1.

In the sequences Table 2 below, CDRs within the V_{H} and V_{L} domains are indicated in boldface as determined by the Chothia approach (Chothia et al. (1992) J. Mol. Biol., 227, 776-798, Tomlinson et al. (1995) EMBO J., 14, 4628-4638 and Williams et al. (1996) J. Mol. Biol., 264, 220-232). See also www2.mrc-lmb.cam.ac.uk/vbase/alignments2.php.

TABLE 2
Amino Acid Sequences of Exemplary
Anti-CD19 Antibodies

| SEQ | | |
|---|---|---|
| ID | | |
| NO: | Description | Amino Acid Sequence |
| 3 | 2018EP187-A12 | QVQLQQWGAGLLKPSETLSL |
| | (a.k.a., | TCAVYGGSFSGYYWTWIRQP |
| | EP187-A12) | PGKGLEWIGEINHGGSSNYN |
| | V_{H} | PSLKSRVTISVDTSKKQFSL |
| | | NLNSVTAADTAVYYCARGLG |
| | | YRSGWYEVENAFDIWGQGTM |
| | | VTVSS |
| 15 | EP187-A12; | GYYWT |
| | V_{H}-CDR1 | |
| 16 | EP187-A12; | EINHGGSSNYNPSLKS |
| | V_{H}-CDR2 | |
| 17 | EP187-A12; | GLGYRSGWYEVENAFDI |
| | V_{H}-CDR3 | |
| 4 | 2018EP187-A12 | QPVLTQPPSVSVAPGQTARI |
| | (a.k.a., | TCGGNKIESRSVHWYQQKPG |
| | EP187-A12) | QAPVLVVYDDGARPSGIPER |
| | V_{L} | LSGSNSGDTATLTISRVEPG |
| | | DEADYYCQVWDGSSVIFGGG |
| | | TKLTVL |
| 18 | EP187-A12; | GGNKIESRSVH |
| | V_{L}-CDR1 | |
| 19 | EP187-A12; | DDGARPS |
| | V_{L}-CDR2 | |
| 20 | EP187-A12; | QVWDGSSVI |
| | V_{L}-CDR3 | |
| 5 | 2018EP188-B10 | QVQLVQSGGGVVQPGKSLRL |
| | (a.k.a., | SCAASGFPFSSYTMHWVRQP |
| | EP188-B10) | PGEGLEWVALISYDGRNLYY |
| | V_{H} | ADSVKGRFTISRDNSYNSLY |
| | | LQLSGLRAEDTALYYCARDI |
| | | NRDHFYGMDLWGPGTTVTVS |
| | | S |
| 21 | EP188-B10; | SYTMH |
| | V_{H}-CDR1 | |
| 22 | EP188-B10; | LISYDGRNLYYADSVKG |
| | V_{H}-CDR2 | |
| 23 | EP 188-B10; | DINRDHFYGMDL |
| | V_{H}-CDR3 | |
| 6 | 2018EP188-B10 | SYELTQPPSVSVAPGQTARI |
| | (a.k.a., | PCGGTNIGSKGVHWYQQKPG |
| | EP188-B10) | QAPVLVIYYDHSRPSGIPER |
| | V_{L} | FSGSNSGNTAALTISRVEAG |
| | | DEADYYCQVWEGTSDHPVFG |
| | | GGTKLTVL |
| 24 | EP188-B10; | GGTNIGSKGVH |
| | V_{L}-CDR1 | |
| 25 | EP188-B10; | YDHSRPS |
| | V_{L}-CDR2 | |
| 26 | EP188-B10; | QVWEGTSDHPV |
| | V_{L}-CDR3 | |
| 7 | 2018EP142-D09 | EVQLVESGAEVKKPGASVKV |
| | (a.k.a., | SCKASGYTFTSYYMHWVRQA |
| | EP142-D09) | PGQGLEWMGIINPSGGSTSY |
| | v_{H} | AQKFQGRVTITADESTSTAY |
| | | MELSSLRSEDTAVYYCAREG |
| | | GYKDFDYWGQGTLVTVSS |
| 27 | EP142-D09; | SYYMH |
| | V_{H}-CDR1 | |
| 28 | EP142-D09; | IINPSGGSTSYAQKFQG |
| | V_{H}-CDR2 | |
| 29 | EP142-D09; | EGGYKDFDY |
| | V_{H}-CDR3 | |
| 8 | 2018EP142-D09 | DIVMTQSPSSLSASVGDRVT |
| | (a.k.a., | VTCRASQSIDTYLNWYQQKP |
| | EP142-D09) | GKAPKLLIYTASTLQSGVPS |
| | V_{L} | RFSGSGSGTDFTLTISSLQP |
| | | EDFATYYCQQSYSAPRTFGQ |
| | | GTKVEIK |
| 30 | EP142-D09; | RASQSIDTYLN |
| | V_{L}-CDR1 | |
| 31 | EP142-D09; | TASTLQS |
| | V_{L}-CDR2 | |
| 32 | EP142-D09; | QQSYSAPRT |
| | V_{L}-CDR3 | |
| 9 | 2018EP188-A1 | QVQLVQSGAEVKKPGASVKV |
| | (a.k.a., | SCKASGYTFTGYYMHWVRQA |
| | EP188-A1) | PGQGLEWMGWINPNSGGTNY |
| | V_{H} | AQKFQGRVTMTRDTSISTAY |
| | | MELSRLRSDDTAVYYCAREA |
| | | LPWDKWYGGYEAFDYWGQGT |
| | | LVTVSS |
| 33 | EP188-A1; | GYYMH |
| | V_{H}-CDR1 | |
| 34 | EP188-A1; | WINPNSGGTNYAQKFQG |
| | V_{H}-CDR2 | |
| 35 | EP188-A1; | EALPWDKWYGGYEAFDY |
| | V_{H}-CDR3 | |
| 10 | 2018EP188-A1 | NIQMTQSPSSLSASIGDRVT |
| | (a.k.a., | ITCRASQGLNTYVAWYQQKP |
| | EP188-A1) | GKAPKLLMYDASTLQSGVPA |
| | V_{L} | RFSGTGSGTDFTLTISSLQP |
| | | EDFATYYCQQVNSFGYTFGQ |
| | | GTKLEIK |
| 36 | EP188-A1; | RASQGLNTYVA |
| | V_{L}-CDR1 | |
| 37 | EP188-A1; | DASTLQS |
| | V_{L}-CDR2 | |
| 38 | EP188-A1; | QQVNSFGYT |
| | V_{L}-CDR3 | |

In some embodiments, the anti-CD19 antibodies described herein bind to the same epitope of a CD19 polypeptide as any of the exemplary antibodies described herein (for example, EP187-A12, EP188-B10, EP142-D09, or EP188-A1) or compete against the exemplary antibody from binding to the CD19 antigen. In some examples, the exemplary antibody is EP187-A12. In other examples, the exemplary antibody is EP188-A1. In yet other examples, the exemplary antibody is EP188-B10. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-CD19 antibody comprises the same V_{H} and/or V_{L} CDRs as an exemplary antibody described herein. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-CD19 antibodies may have the same V_{H}, the same V_{L}, or both as compared to an exemplary antibody described herein.

Also within the scope of the present disclosure are functional variants of any of the exemplary anti-CD19 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of CD19 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-CD19 antibody may comprise a V_{H} fragment having CDR amino acid residue variations in only the heavy chain CDR1 as compared with the V_{H} of the exemplary antibody. In some examples, the anti-CD19 antibody may further comprise a V_{L} fragment having the same V_{L} CDR3, and optionally same V_{L} CDR1 or V_{L} CDR2 as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring

Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CD19 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-CD19 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-CD19 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD19 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-CD19 antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus). Exemplary scFv anti-CD19 antibodies are provided below in Table 3 (CDRs in boldface and peptide linker in boldface and underlined):

TABLE 3
Exemplary scFv Anti-CD19 Antibodies
2018EP187-Al2 (scFv, V_{H}-V_{L} orientation)

QVQLQQWGAGLLKPSETLSLTCAVYGGSFSGYYWT
WIRQPPGKGLEWIGEINHGGSSNYNPSLKSRVTIS
VDTSKKQFSLNLNSVTAADTAVYYCARGLGYRSGW
YEVENAFDIWGQGTMVTVSS [symbol] [symbol]
QPVLTQPPSVSVAPGQTARITCGGNKIESRSVHWY
QQKPGQAPVLVVYDDGARPSGIPERLSGSNSGDTA
TLTISRVEPGDEADYYCQVWDGSSVIFGGGTKLTV
L (SEQ ID NO: 11)
2018EP188-B10 (scFv, V_{H}-V_{L} orientation)
QVQLVQSGGGVVQPGKSLRLSCAASGFPFSSYTMH
WVRQPPGEGLEWVALISYDGRNLYYADSVKGRFTI
SRDNSYNSLYLQLSGLRAEDTALYYCARDINRDHF
YGMDLWGPGTTVTVSS [symbol] YEL
TQPPSVSVAPGQTARIPCGGTNIGSKGVHWYQQKP
GQAPVLVIYYDHSRPSGIPERFSGSNSGNTAALTI
SRVEAGDEADYYCQVWEGTSDHPVFGGGTKLTVL
(SEQ ID NO: 12)
2018EP142-D09 (scFv, V_{H}-V_{L} orientation)
EVQLVESGAEVKKPGASVKVSCKASGYTFTSYYMH
WVRQAPGQGLEWMGIINPSGGSTSYAQKFQGRVTI
TADESTSTAYMELSSLRSEDTAVYYCAREGGYKDF
DYWGQGTLVTVSS [symbol] DIVMTQS
PSSLSASVGDRVTVTCRASQSIDTYLNWYQQKPGK
APKLLIYTASTLQSGVPSRFSGSGSGTDFTLTISS
LQPEDFATYYCQQSYSAPRTFGQGTKVEIK
(SEQ ID NO: 13)
2018EP188-A1 (scFv, VH-V_{L} orientation
QVQLVQSGAEVKKPGASVKVSCKASGYTFTGYYMH
WVRQAPGQGLEWMGWINPNSGGTNYAQKFQGRVTM
TRDTSISTAYMELSRLRSDDTAVYYCAREALPWDK
WYGGYEAFDYWGQGTLVTVSS [symbol]
NIQMTQSPSSLSASIGDRVTITCRASQGLNTYVA
WYQQKPGKAPKLLMYDASTLQSGVPARFSGTGSGT
DFTLTISSLQPEDFATYYCQQVNSFGYTFGQGTKL
EIK (SEQ ID NO: 14)

Any of the anti-CD19 antibody as described herein, e.g., the exemplary anti-CD19 antibodies provided here, can bind and inhibit (e.g., reduce or eliminate) the activity of CD19-positive cells (e.g., B cells). In some embodiments, the anti-CD19 antibody as described herein can bind and inhibit the activity of CD19-positive cells by at least 30% (e.g., 35%, 40%, 45%, 50%, 60%, 70%, 80%, 90%, 95% or greater, including any increment therein). The inhibitory activity of an anti-CD19 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{\begin{matrix}( [ E ] - [ I ] - {K}_{i}^{a p p} ) + \\ \sqrt{{( [ E ] - [ I ] - {K}_{i}^{a p p} )}^{2} + 4 [ E ]} \cdot {K}_{i}^{a p p}\end{matrix}}{( 2 )} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-CD19 antibody described herein may have a Ki^{app} value of 1000, 500, 100, 50, 40, 30, 20, 10, 5 pM or less for the target antigen or antigen epitope.

In some embodiments, any of the anti-CD19 antibodies disclosed herein (e.g., EP187-A12, EP188-A01, EP188-B10, or EP142-D9) may be a bispecific antibody, which may further comprise a binding moiety specific to a second (non-CD19) antigen. In some examples, the exemplary antibody is EP187-A12. In other examples, the exemplary antibody is EP188-A1. In yet other examples, the exemplary antibody is EP188-B10. In some examples, the bispecific antibody can be a bispecific T cell engager (BiTE) capable of binding to CD19 and a T cell biomarker, for example, CD3. In other examples, the bispecific antibody can bind to CD19 and a biomarker of an immune cell, for example, NK cell, macrophage, etc. Such a bispecific antibody can engage immune cells to CD19⁺ disease cells such as cancer cells, thereby eliciting immune responses against the CD19⁺ disease cells.

In some instances, the bispecific antibodies disclosed herein may comprise a first binding moiety comprising the same heavy chain and light chain CDRs or the same V_{H} and V_{L} fragments as one of the exemplary anti-CD19 antibodies (e.g., EP187-A12, EP188-A01, EP188-B10, or EP142-D9) and a second binding moiety specific to a biomarker of an immune cell, e.g., T cell, NK cell, macrophage, etc. Exemplary anti-CD19/anti-CD3 bispecific antibodies are provided in Examples below.

### II. Preparation of Anti-CD19 Antibodies

Antibodies capable of binding CD19 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-CD19 antibody may be identified from a suitable library (e.g., a human antibody library).

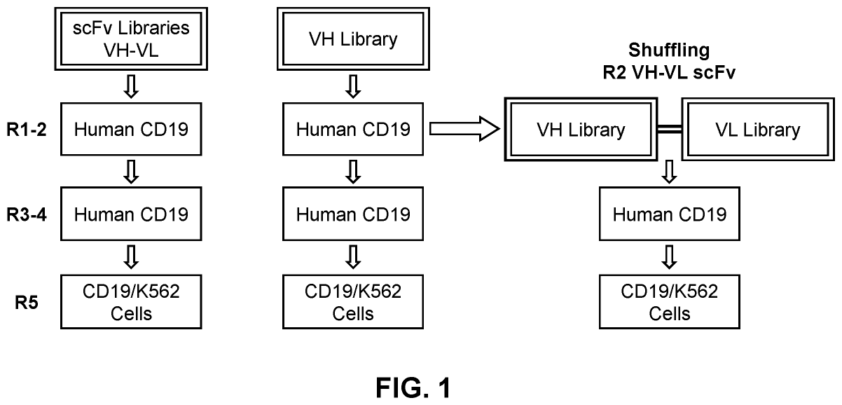

In some instances, high affinity fully human CD19 binders may be obtained from a human antibody library following the screening strategy illustrated in FIG. 1. See also Example 1 below. This strategy allows for maximizing the library diversity to cover board and active epitopes on CD19 expressing cells.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in enhancing the activity of CD19. It will be apparent to one of skill in the art that one or more polynucleotide changes can be made to the antibody and still maintain its binding specificity to the target antigen.

Alternatively, antibodies capable of binding to the target antigens as described herein (a CD19 molecule) may be isolated from a suitable antibody library via routine practice. Antibody libraries can be used to identify proteins that bind to a target antigen (e.g., human CD19 such as cell surface CD19) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to CD19 can be identified from the library following routine procedures. Positive clones can be subjected to further screening to identify those that enhance CD19 activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of CD19 have been replaced (swapped) with sequences from a closely related, but antigenically distinct protein (such as another member of the tumor necrosis factor receptor family). By assessing binding of the antibody to the mutant CD19, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-CD19 antibody is prepared by recombinant technology as exemplified below.

Nucleic acids encoding the heavy and light chain of an anti-CD19 antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-CD19 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-CD19 antibody and the other encoding the light chain of the anti-CD19 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-CD19 antibody as described herein, vectors (e.g., expression vectors) containing such; and host cells comprising the vectors are within the scope of the present disclosure.

### Applications of Anti-CD19 Antibodies

Any of the anti-CD19 antibodies disclosed herein can be used for therapeutic, diagnostic, and/or research purposes, all of which are within the scope of the present disclosure.

Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying CD19⁺ disease cells. Examples of such target diseases/disorcers include hematopoietic cancers, e.g., a cancer of B cell lineage. Examples include, but are not limited to, hematological B cell neoplasms including lymphocytic leukemia, e.g., B Cell chronic lymphocytic leukemia (CLL); B-cell acute lymphoblastic leukemia (ALL), and B-cell non-Hodgkin's lymphoma (NHL).

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the anti-CD19 antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Kits for Use in Treatment of Diseases

The present disclosure also provides kits for use in treating or alleviating a target disease, such as hematopoietic cancer as described herein. Such kits can include one or more containers comprising an anti-CD19 antibody, e.g., any of those described herein. In some instances, the anti-CD19 antibody may be co-used with a second therapeutic agent.

In some embodiments, the kit can comprise instructions for use in accordance with any of the methods described herein. The included instructions can comprise a description of administration of the anti-CD19 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-CD19 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-CD19 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Generation of Fully Human Anti-CD19 Antibodies

Fully human antibodies having binding specificity to cell-surface human CD19 were identified from a human antibody library as follows.

### Generation of CD19 Overexpression Recombinant Cell Lines

HEK293 and K562 cells (ATCC) were transfected with a pCMV6-Entry vector carrying a nucleotide sequence encoding the full-length human CD19 fused with flag and Myc tags at the C-terminus. G418 drug selection process yielded a polyclonal, drug resistant pool of CD19-expressing cells. In parallel, the parental cell line transferred with the empty pCMV6-Entry vector was generated for use as a negative control. The CD19-expressing cells were sorted by FACS to yield a pool of CD19-expressing cells. The pool was expanded under G418 drug selection. Single cell sorting was then performed followed by further drug selection to generate clonal cell lines. The clonal lines were screened for CD19 expression by FACS. The cell line showing a high expression level of CD19 was selected for use in selection, screening and assays as disclosed herein.

Screening for Anti-CD19 Antibodies from a Human Antibody Libraries

Natural human antibody libraries were constructed from bone marrow MNCs and PBMCs of multiple naïve health donors and autoimmune disease patient doners. RT-PCR was performed to capture the full immunoglobulin repertoire of both V_{H} and V_{L} domains (producing V_{H} and V_{L} libraries). A single-chain antibody (scFv) library was then constructed by V_{H} and V_{L} shuffling. The library size is predicted to be 10¹²⁻¹³. The V_{H} and scFv libraries have been further modified to insert in vitro transcription and translation signals at the N-terminus and a flag tag to the C-terminus of the antibody fragment, respectively, for selection by mRNA display.

mRNA display technology was then used for the identification of CD19 binders from the above constructed V_{H} and scFv libraries following conventional practice (see, e.g., U.S. Pat. No. 6,258,558B1, the relevant disclosures of which are incorporated by reference herein for the subject matter or purpose referenced herein. Briefly, the DNA libraries were first transcribed into mRNA libraries and then translated into mRNA-V_{H} or scFv fusion libraries by covalent coupling through a puromycin linker. The libraries were then purified and converted to mRNA/cDNA fusion libraries. The fusion libraries were first counter selected with human IgGs (negative selection) or K562 cells to remove non-specific binders, followed by selection against either recombinant CD19-Fc fusion protein captured on Protein G magnetic beads (round 1-3) or on CD19 overexpression recombinant K562 cells (rounds 4-5). The CD19 binders were recovered and enriched by PCR amplification. At round 3, enriched V_{H} library was converted to scFv library by shuffling with a naïve V_{L} library noted above and further enriched for 3 more rounds. A total of 5 rounds of selections was executed to generate highly enriched anti-CD19 antibody pools, as illustrated in FIG. 1.

The enriched anti-CD19 antibody pools were cloned into the bacterial periplasmic expression vector pET22b, which was transformed into TOP 10 competent cells. Each of the scFv molecules was engineered to have a C-terminal flag and 6×HIS tag for purification and assay detection. Clones from TOP 10 cells were pooled and the miniprep DNA were prepared and subsequently transformed into bacterial Rosetta II strain for expression. Single clone was picked, grown and induced with 0.1 mM IPTG in 96 well plate for expression. The supernatant was collected after 16-24 hours induction at 30° C. for assays to identify anti-CD19 antibodies.

The supernatant samples were assessed with sandwich ELISA assay to determine the presence/level of the anti-CD19 scFv antibody contained therein. Briefly, a 96 well plate was immobilized with anti-HIS tag antibody (R&D Systems) at a final concentration of 2 μg/mL in 1×PBS in a total volume of 50 μL per well. The plate was incubated overnight at 4° C. followed by blocking with 200 μL per well of a superblock buffer for 1 hour. 100 μl of 1:10 1×PBST diluted supernatant were added to each well and incubated for 1 hour with shaking. The expression level of the CD19 scFv was detected by incubating the mixture in the plate with 50 μL of an HRP-conjugated anti-Flag antibody, which is diluted at 1:5000 in 1×PBST, for one hour. In between each step, the plate was washed 3 times with 1×PBST in plate washer. The plate was then developed with 50 μl of the TMB substrate for 5 mins and stopped by adding 50 μl of 2N sulfuric acid. The plate was read at OD450 nm in Biotek plate reader and the data was analyzed with Excel bar graph.

CD19 binding screening ELISA was developed to identify individual CD19 binders. Briefly, 96 well plate was immobilized with a human Fc as a control or a human CD19-Fc protein at final concentration of 2 μg/mL in 1×PBS in a total volume of 50 μL per well. The plate was incubated overnight at 4° C. followed by blocking with 200 μL per well of a superblock buffer for 1 hour. 100 μl of the supernatant was added to each of the Fc and CD19-Fc fusion protein immobilized wells and incubated for 1 hour with shaking. The CD19 binding was detected by adding a 50 μL of HRP-conjugated anti-Flag antibody, which was diluted at 1:5000 in 1×PBST. In between each step, the plate was washed 3 times with 1×PBST in a plate washer. The plate was then developed with 50 μl of the TMB substrate for 5 mins and stopped by adding 50 μl of 2N sulfuric acid. The plate was read at OD450 nm Biotek plate reader and the binding and selectivity was analyzed with Excel bar graph.

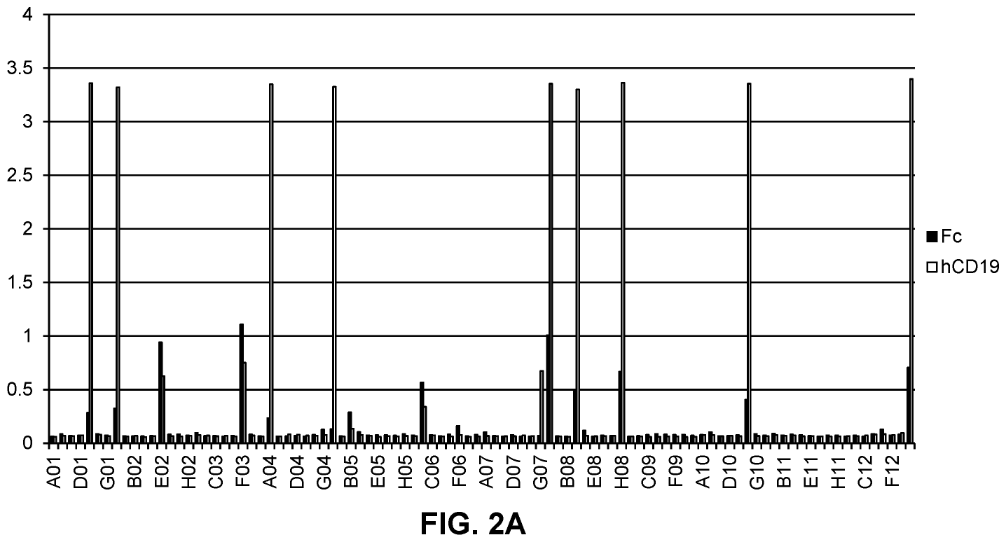

A number of positive anti-CD19 clones was identified in the screening process disclosed herein as exemplified in FIG. 2.

### Example 2. Identification of Exemplary Anti-CD19 Clones Capable of Binding to Cell Surface-Expressed CD19

Production and Purification of Anti-CD19 Antibodies in E. coli Cells

Cells expressing V_{H} or anti-CD19 scFv antibodies identified in the screening process disclosed in Example 1 above were picked from a glycerol stock plate and grown overnight into a 5 mL culture in a Thomson 24-well plate with a breathable membrane. Bacterial cells as described in the Examples herein were grown at 37° C. and shaking at 225 RPM in Terrific Broth Complete plus 100 μg/mL carbenicillin and 34 μg/mL chloramphenicol, with 1:5,000 dilution of antifoam-204 also added, unless specified otherwise. The overnight starter culture was then used to inoculate a larger culture at a suitable dilution rate of starter culture into the designated production culture (e.g., 50 mL culture in 125 mL Thomson Ultra Yield flask, 100 mL culture in 250 mL Ultra Yield Thomson flask or 250 mL culture in 500 mL Ultra Yield Thomson flask) and grown until the OD₆₀₀ was between 0.5-0.8. At this point, the culture was induced with a final concentration of IPTG at 0.5 mM for V_{H} and 0.1 mM scFv and incubated over night at 30° C. The cultures were then spun for 30 min at 5,000×g, to pellet the cells and the supernatant was filter sterilized through a 0.2 μm sterilizing PES membrane for further analysis.

To purify the antibody fragment, 3 μl GE Ni Sepharose Excel resin were mixed with 1 mL of filtered supernatant and loaded onto 10 mL or 20 mL BioRad Econo-Pac columns. Before loading, the resin of the column was equilibrated with at least 20 column volume (CV) buffer A (1×PBS, pH7.4 with extra NaCl added to 500 mM). The filter sterilized supernatant was purified by gravity flow via either controlling the flow to 1 mL/min or being poured over two times, over the same packed resin bed. The column was then washed with the following buffers: 10 CV buffer A, 20CV buffer B (1×PBS, pH7.4 with extra NaCl to 500 mM, and 30 mM imidazole). The two Detox buffers were used to remove endotoxin, if needed. To purify the antibody fragment from the 250 mL expression culture, antibody-bound column was washed sequentially with 20CV buffer C (1×PBS pH7.4 with extra NaCl to 500 mM, 1% Tx114), 20CV buffer D (1× PBS pH7.4 with extra NaCl to 500 mM, 1% Tx100+0.2% TNBP) and 40CV buffer E (1×PBS pH7.4 with extra NaCl to 500 mM).

The protein was eluted with Eluting buffer F (1×PBS pH7.4 with extra NaCl to 500 mM, and 500 mM imidazole) in a total of six fractions (0.5CV pre elute, 5×1CV elute). Fractions were run on a Bradford assay (100 ul diluted Bradford solution+10 ul sample). Fractions with bright blue color were pooled and the protein concentration thereof was measured by A280 extension coefficient. SDS-PAGE gel assay was performed to analyze the purity of the purified antibodies.

In most cases, Tm shift thermal stability assay was performed to measure the thermal stability of the purified antibodies.

### Cell Surface Binding Activity of Anti-CD19 ScFv Antibody by FACS Analysis

To determine the binding EC50 value of each anti-CD19 antibody to cell surface-expressed CD19, each purified scFv protein was titrated from 100 nM with 2-fold serial dilutions in full medium. The diluted samples were incubated with CD19-expressing HEK293 cells (CD19/HEK293 cells) in 96 wells plate on ice for 1 hour. Cells were spun down at 1200 rpm for 5 minutes at 4° C. to remove unbound antibodies. Cells were then washed once with 200 μL of full medium per well. Samples were mixed with an Alexa fluor 488-conjugated anti-His antibody (secondary antibody, 100 μL, 1:1000 diluted) and incubated at 4° C. for 30 minutes in dark. Samples were then spun down at 1200 rpm for 5 minutes at 4° C. and washed twice with 200 uL of 1×PBS per well. The resultant samples were reconstituted in 200 uL of 1×PBS and read on Guava EasyCyte. Analysis was done by counting only Alexa Fluor 488-positive cells and then plotted in Prism 8.1 software.

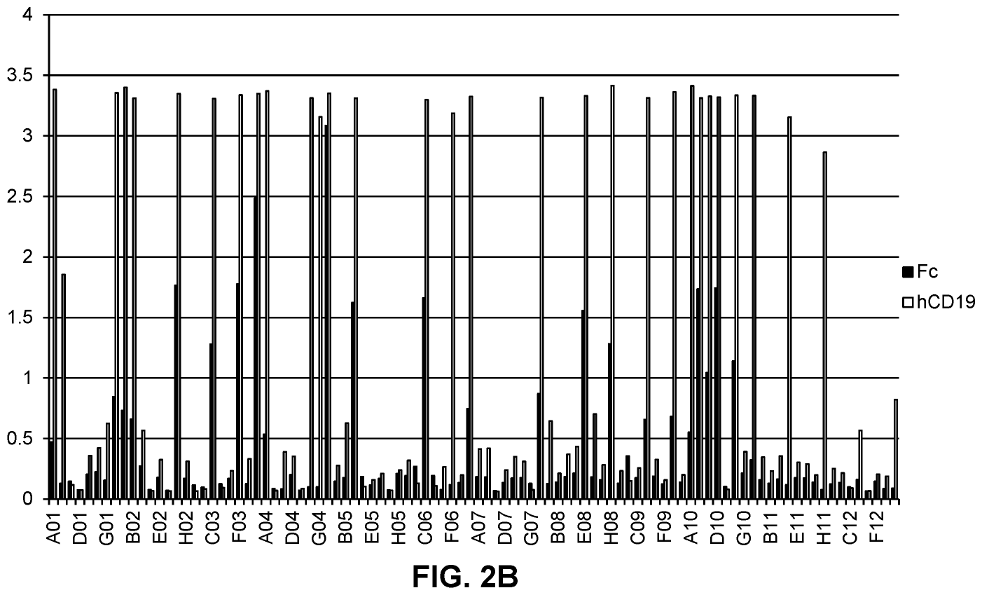

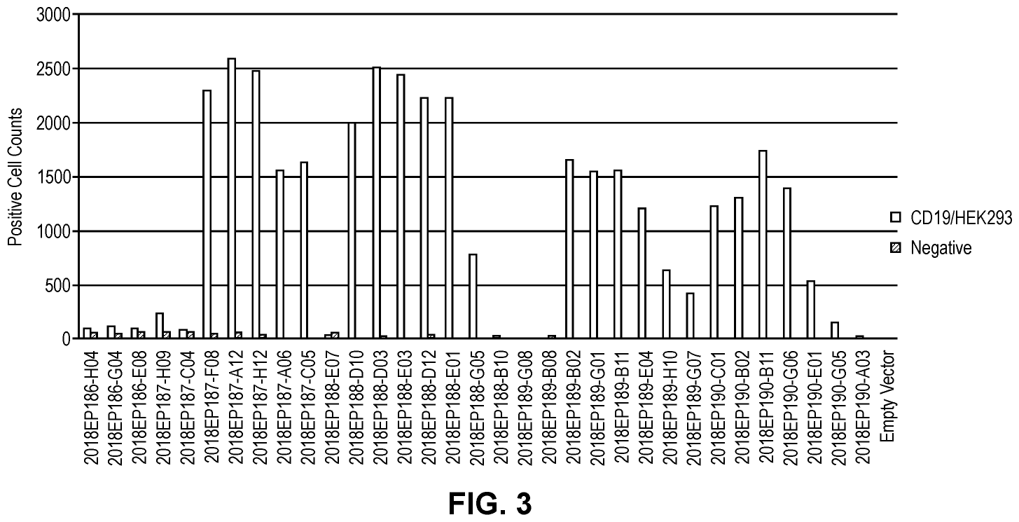

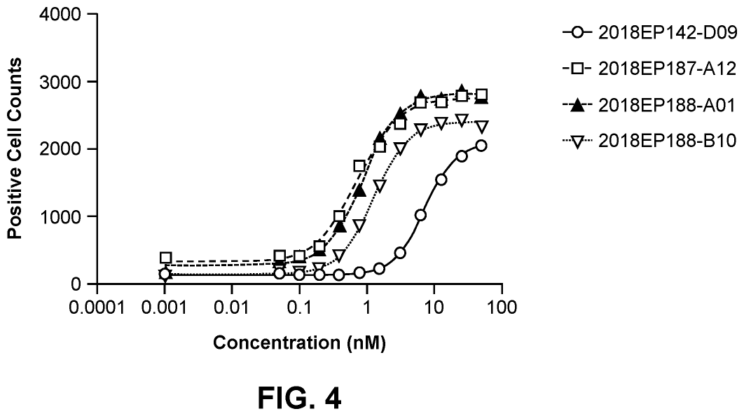

Exemplary anti-CD19 clones capable of binding to cell surface CD19 as determined in this study were shown in FIG. 3. FIG. 4 shows binding curves of four exemplary anti-CD19 clones, EP142-D09, EP187-A12, EP188-A01, and EP188-B10, at various concentrations as indicated. EC50 values of these exemplary anti-CD19 antibodies are provided in Table 4 below:

TABLE 4
EC50 Values of Exemplary Anti-CD19 Antibodies

| | EP142-D09 | EP187-A12 | EP188-A01 | EP188-B10 |
|---|---|---|---|---|
| EC50 (nM) | 7.177 | 0.7515 | 0.864 | 1.264 |

At least EP187-A12, EP188-A01, and EP188-B10 showed better binding affinity to cell surface CD19 than FMC63 (the EC50 value of which was found to be around 15 nM measured in the same assay).

### Example 3. Epitope Binning of Anti-CD19 ScFv Antibodies

Purified anti-CD19 scFv antibodies were titrated from 100 nM with 2-fold serial dilution. Each diluted sample was mixed with 20 nM of the anti-CD19 FMC63 IgG antibody and then incubated with CD19/HEK293 cells for 1 hour at 4° C. Cells were spun down at 1200 rpm for 5 minutes at 4° C. Samples were mixed with an Alexa fluor 488-conjugated anti-His antibody (100 uL, 1:1000 diluted) and incubated at 4° C. for 30 minutes in the dark. The samples were spun down at 1200 rpm for 5 minutes at 4° C. and washed twice with 200 μL of 1×PBS per well. The resultant samples were then reconstituted in 200 uL of 1×PBS and read on Guava EasyCyte. Analysis was done by counting only Alexa Fluor 488-positive cells and then plotted in Prism 8.1 software.

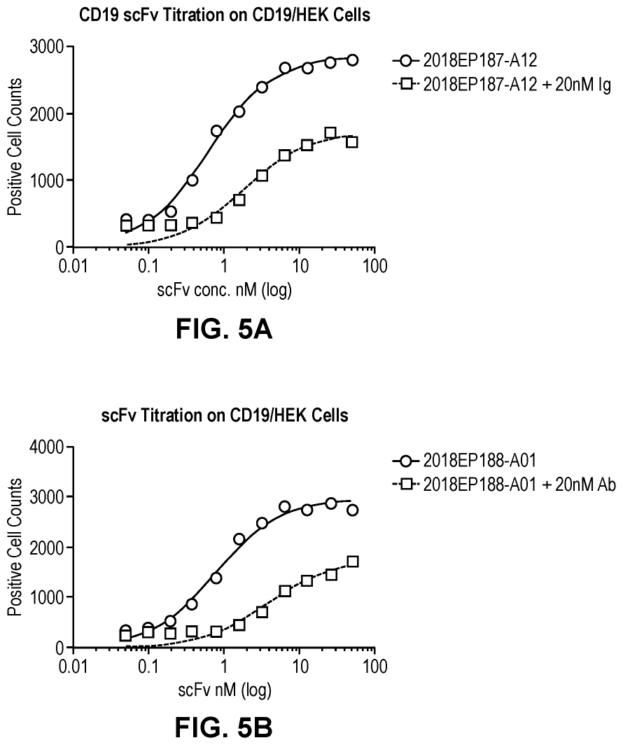

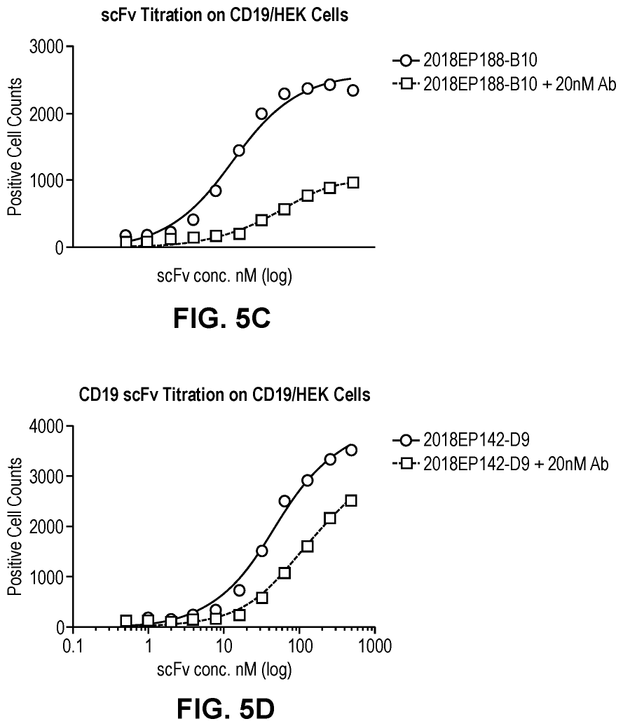

As shown in FIGS. 5A-5D, the anti-CD19 FMC63 IgG antibody did not fully compete against the exemplary anti-CD19 antibody clones EP142-D09, EP187-A12, EP188-A01, and EP188-B10 from binding to CD19/HEK293 cells. The result indicates that the exemplary anti-CD19 antibodies do not appear to bind to the same CD19 epitope as FMC63.

### Example 4. Thermal Stability Assessment of Exemplary Anti-CD19 scFv Antibodies

In this example, each sample and control were prepared in at least a duplicate to make sure the results were reproducible. A plate map was designed first in Excel so the exact location of each sample can be matched to the software for running and analyzing the samples.

A fresh dilution of Protein Thermal Shift Dye (1000×) to 8× was prepared in water. A MicroAmp Optical 96 well plate or 8 cap stripe by LifeTech were used for the experiments. The following reagents were added in the order listed:

1^{st} sample: 5 ul Protein Thermal Shift Buffer,

2^{nd} sample: 12.5 ul sample diluted to 0.4 mg/mL in water,

3^{rd} sample: 2.5 ul diluted Thermal Shift Dye 8× for a total volume of 20 ul/well.

Negative control sample: 12.5 ul buffer with no protein

Positive control sample: 10.5 ul water with 2.0 uL Protein Thermal Shift Control Protein.

The Thermal shift dye, once added, was pipetted up and down for 10 times. The plates or strips were then spun down for 1000 RPM for 1 min once sealed with MicroAmp Optical film of caps. Afterwards, the plate or strips was put into a Quant Studio 3 instrument by Thermo Fisher with the proceeding method being run as follows.

Step 1: 100% ramp rate to 25.0° with time 2 min

Step 2: 1% ramp rate to 99.0° C. with time 2 min

The samples and subsequent Tm were then analyzed (and Tm calculated) using the QuantStudio Design and Analysis Software and the Protein Thermal Shift Software 1.3. The results are shown in Table 5 below:

TABLE 5
Thermal Shift Assay of Exemplary Anti-CD19 Antibodies

| Clone Name | Tm ° C. |
|---|---|
| 2018EP187-A12 | 67.41 |
| 2018EP188-B10 | 66.6 |
| 2018EP142-D09 | 59.5 |

At least clones EP187-A12 and EP188-B10 showed better thermostability of FMC63, which showed a Tm value of around 59° C. in the same assay.

### Example 5: Anti-CD19 Antibodies Bind to Endogenous CD19 and Recombinant CD19 on Cell Surface

Exemplary anti-CD19 scFv antibodies, including EP187-A12, EP188-B10, EP142-D09, and EP188-A01, were tested for their ability to bind to endogenous CD19 expressed on cell surface and recombinant CD19 expressed on cell surface using FACS.

Briefly, 200 nM of each of purified anti-CD19 scFv antibodies (containing a HIS tag) were diluted in full medium and incubated with Daudi cells and Raji cells, CD19/HEK293, CD19/K562, and K562 cell lines in 96 wells plate on ice for 1 hour. Cells were spun down at 1200 rpm for 5 minutes at 4° C. to remove unbound scFvs. Cells were then washed once with 200 uL of full medium per well. Samples were detected with anti-HIS biotin/Streptavidin Alexa fluor 647 by adding 100 uL of diluted secondary antibody and incubated at 4 C for 30 minutes in the dark. Samples were spun down at 1200 rpm for 5 minutes at 4° C. and washed twice with 200 uL of 1×PBS per well. The samples were reconstituted in 200 uL of 1×PBS and read on Attune NxT cytometer. Analysis was done by Attune NxT software plotting the overlay histogram of CD19 proteins binding onto both negative and target cell lines. FMC63 scFv Ab and anti-HIS biotin/Streptavidin secondary Alexa fluor 647 as positive and negative (background) controls for the assay.

As indicated in FIG. 7, all four CD19 scFv antibodies bind to HEK and K562 expressing recombinant CD19 on cell surface at the tested antibody concentration. EP187-A12 and EP188-B10 were found to bind to Daudi and Raji cells expressing endogenous CD19 as well.

Further, immunohistochemistry (IHC) studies were performed with 5-mm sections from formalin-fixed, paraffin-embedded diffuse large B cell lymphoma (DLBCL) FFPE tissue block performed on Ventana Ultra automation platform using IHC staining protocol. Briefly, after deparaffinization and rehydration, the antigen retrieval was performed with standard CC1 antibody retrieval (EDTA based antigen retrieval buffer, pH 9.0, Cat #950-500). The tissue permeabilized and washed with Ventana discovery wash, Cat #905-510 and discovery reaction buffer, Cat #950-300 between staining steps. Discovery inhibitor CM Cat #764-4307 and IHC/ICC IHC protein blocker (Invitrogen Cat #00-4952-54) pretreatment for non-specific staining were applied during staining.

Exemplary anti-CD19 scFv EP187-A12, fused to a human Fc fragment, was incubated with the tissue samples noted above at a concentration of 10 ug/ml for 60 min at 37° C., followed by incubation with Anti-Human IgG FC HRP antibody at 1/250 dilution (Abcam Cat #ab98624). Ventana ChromapDAB kit (Cat #760-159) was used for final IHC steps. All the sections were counterstained with hematoxylin, and the whole slide imaged by Aperio AT2 scanscope and image analysis with Indica labs CytoNuclear v1.6 Algorithms.

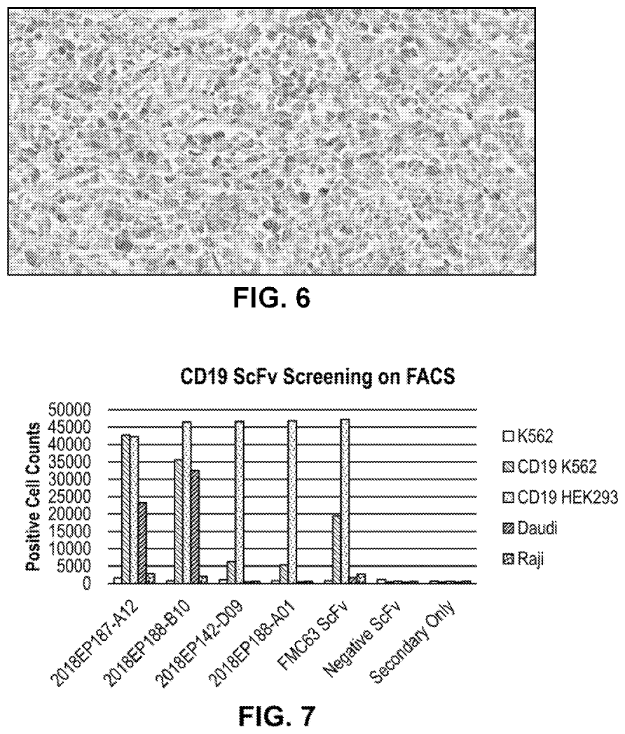

As shown in FIG. 6, EP187-A12 was found to bind to CD19-positive DLBCL tissue in the IHC study provided herein, indicating that this antibody is capable of binding to endogenous CD19, which may be expressed by disease cells.

### Example 6: Expression and Purification of Anti-CD19/Anti-CD3 BiTE Antibodies

This example describes the generation of BiTE bispecific antibodies having binding specificity for CD-19 and CD-3. These antibodies find use as therapeutic antibodies.

For BiTE generation, the anti-CD19 ScFv antibodies in V_{H}-V_{L} order of sequences were fused to an anti-CD3 antibody in V_{H}-V_{L} order of ScFv format through a (G4S) linker. A 6×His tag was directly added to the C-terminus of the BiTE. The DNA sequences corresponding to the BiTE antibodies were codon optimized for mammalian expression, synthesized and subcloned to pCDNA3.4 expression vector.

Four BiTE antibodies were constructed, namely EP381, EP382, EP383 and EP384. The sequences of these molecules is shown below (annotated following each sequence):

BiTE EP381 (SEQ ID NO: 39; SEQ ID NO: 40 for the BiTE with no
signal peptide and His-tag)
METDTLLLWVLLLWVPGSTGQVQLQQWGAGLLKPSETLSLTCAVYGGSFSGYYWTWIRQPPGKGLEWIGEINHGGSS
NYNPSLKSRVTISVDTSKKQFSLNLNSVTAADTAVYYCARGLGYRSGWYEVENAFDIWGQGTMVTVSSGGGGSGGGG
SGGGGSQPVLTQPPSVSVAPGQTARITCGGNKIESRSVHWYQQKPGQAPVLVVYDDGARPSGIPERLSGSNSGDTAT
LTISRVEPGDEADYYCQVWDGSSVIFGGGTKLTVLGGGGSEVQLVESGGGLVQPGGSLRLSCAASGFTFSTYAM
NWVRQAPGKGLEWVGRIRSKYNNYATYYADSVKGRFTISRDDSKNTLYLQMNSLRAEDTAVYYCVRHGNF
GDSYVSWFAYWGQGTLVTVSSGGGGSGGGGSGGGGSGGGGS

[symbol]

[symbol]

[symbol]

[symbol]

[symbol]

[symbol]

[symbol]

[symbol]

[symbol]

HHHHHH
Signal peptide (SEQ ID NO: 41): italicized
scFv of EP187-Al2 (SEQ ID NO: 11): underlined
VH of anti-CD3 (SEQ ID NO: 42): in boldface
VL of anti-CD3 (SEQ ID NO: 43): in boldface and italicized
BiTE EP382 (SEQ ID NO: 44; SEQ ID NO: 45 for the BiTE with no
signal peptide and His-tag)
METDTLLLWVLLLWVPGSTGQVQLVQSGGGVVQPGKSLRLSCAASGFPFSSYTMHWVRQPPGEGLEWVALISYDGRN
LYYADSVKGRFTISRDNSYNSLYLQLSGLRAEDTALYYCARDINRDHFYGMDLWGPGTTVTVSSGGGGSGGGGSGGG
GSSYELTQPPSVSVAPGQTARIPCGGTNIGSKGVHWYQQKPGQAPVLVIYYDHSRPSGIPERFSGSNSGNTAALTIS
RVEAGDEADYYCQVWEGTSDHPVFGGGTKLTVLGGGGSEVQLVESGGGLVQPGGSLRLSCAASGFTFSTYAMNW
VRQAPGKGLEWVGRIRSKYNNYATYYADSVKGRFTISRDDSKNTLYLQMNSLRAEDTAVYYCVRHGNFGD
SYVSWFAYWGQGTLVTVSSGGGGSGGGGSGGGGSGGGGS

[symbol]

[symbol]

[symbol]

[symbol]

[symbol]

[symbol]

[symbol]

[symbol]

[symbol]

[symbol]

[symbol]

HHHHHH
Signal peptide (SEQ ID NO: 41): italicized
scFv of EP188-B10 (SEQ ID NO: 12): underlined
VH of anti-CD3 (SEQ ID NO: 42): in boldface
VL of anti-CD3 (SEQ ID NO: 43): in boldface and italicized
BiTE EP383 (SEQ ID NO: 46; SEQ ID NO: 47 for the BiTE with no
signal peptide and His-tag)
METDTLLLWVLLLWVPGSTGEVQLVESGAEVKKPGASVKVSCKASGYTFTSYYMHWVRQAPGQGLEWMGIINPSGGS
TSYAQKFQGRVTITADESTSTAYMELSSLRSEDTAVYYCAREGGYKDFDYWGQGTLVTVSSGGGGSGGGGSGGGGSD
IVMTQSPSSLSASVGDRVTVTCRASQSIDTYLNWYQQKPGKAPKLLIYTASTLQSGVPSRFSGSGSGTDFTLTISSL
QPEDFATYYCQQSYSAPRTFGQGTKVEIKGGGGSEVQLVESGGGLVQPGGSLRLSCAASGFTFSTYAMNWVRQ
APGKGLEWVGRIRSKYNNYATYYADSVKGRFTISRDDSKNTLYLQMNSLRAEDTAVYYCVRHGNFGDSYV
SWFAYWGQGTLVTVSSGGGGSGGGGSGGGGSGGGGS

[symbol]

[symbol]

[symbol]

[symbol]

[symbol]

[symbol]

[symbol]

[symbol]

[symbol]

[symbol]

[symbol]

[symbol]

[symbol]

HHHHHH
Signal peptide (SEQ ID NO: 41):
italicized
scFv of EP142-D09 (SEQ ID NO: 13):
underlined
VH of anti-CD3 (SEQ ID NO: 42):
in boldface
VL of anti-CD3 (SEQ ID NO: 43):
in boldface and italicized
BiTE EP384 (SEQ ID NO: 48; SEQ ID NO: 49
for the BiTE with no signal peptide and His-tag)
METDTLLLWVLLLWVPGSTGQVQLVQSGAEVKKPGASVKVSCKASGYTFTGYYMHWVRQAPGQGLEWMGWINPNSGG
TNYAQKFQGRVTMTRDTSISTAYMELSRLRSDDTAVYYCAREALPWDKWYGGYEAFDYWGQGTLVTVSSGGGGSGGG
GSGGGGSNIQMTQSPSSLSASIGDRVTITCRASQGLNTYVAWYQQKPGKAPKLLMYDASTLQSGVPARFSGTGSGTD
FTLTISSLQPEDFATYYCQQVNSFGYTFGQGTKLEIKGGGGSEVQLVESGGGLVQPGGSLRLSCAASGFTFSTY
AMNWVRQAPGKGLEWVGRIRSKYNNYATYYADSVKGRFTISRDDSKNTLYLQMNSLRAEDTAVYYCVRHG
NFGDSYVSWFAYWGQGTLVTVSSGGGGSGGGGSGGGGSGGGGS

[symbol]

[symbol]

[symbol]

[symbol]

[symbol]

[symbol]

[symbol]

[symbol]

[symbol]

[symbol]

[symbol]

HHHHHH
Signal peptide (SEQ ID NO: 41):
italicized
scFv of EP188-A1 (SEQ ID NO: 14):
underlined
VH of anti-CD3 (SEQ ID NO: 41):
in boldface
VL of anti-CD3 (SEQ ID NO: 43):
in boldface and italicized

The antibodies were expressed transiently in ExpiHEK293-F cells in free style system (Invitrogen) according to standard protocol. The cells were grown for five days before harvesting. The supernatant was collected by centrifugation and filtered through a 0.2 μm PES membrane. The BiTE antibodies were purified by Ni-Sepharose (GE Healthcare) affinity column according to the manufacturer's protocol. The antibodies were further purified by a Sephadex 200 Increase 10/300 GL column in AKTA for size exclusion chromatographic column purification. The final purified antibodies have endotoxin of less than 10 EU/mg and kept in 1×PBS buffer.

### Example 7: Binding of Anti-CD19/Anti-CD3 BiTE Antibodies to CD19⁺ Raji and CD3⁺ Jurkat Cells

This example evaluates the binding activity of exemplary anti-CD19/anti-CD3 bispecific antibodies to CD19+ and CD3+ cells.

(a) Binding Activity to CD19+ Cells

A solution containing 200 nM of purified anti-CD19/CD3 BiTE antibodies were serial diluted in full medium and incubated with Raji cells in 96 wells plate on ice for one hour. Cells were spun down at 1200 rpm for 5 minutes at 4° C. to remove primary antibodies. Cells were then washed once with 200 uL of full medium per well. Samples were detected with premixed anti-His Biotin Streptavidin Alexa fluor 647 by adding 100 uL of diluted secondary antibody and incubated at 4° C. for 30 minutes in the dark. Samples were spun down at 1200 rpm for 5 minutes at 4° C. and washed twice with 200 uL of 1×PBS per well. Reconstituted samples in 200 uL of 1× PBS and read on Attune NxT cytometer. Analysis was done by Attune NxT software plotting the overlay histogram of CD19 or CD3 binding with secondary antibodies.

The tested bispecific antibodies showed high binding activity to CD19⁺ Raji cells. Around 92.27%, 81.11%, 79.99%, and 92.01% Raji cells were stained positive when incubated with EP381, EP382, EP383, and EP384, respectively. Only 1.3% Raji cells were stained positive when included with a secondary antibody control and a positive control antibody showed 97.1% positive staining Table 6 below provides the EC50 values of the four BiTE antibodies as measured by FACS analysis.

TABLE 6
EC50 Values of BiTE Antibodies Measured by FACS

| | FACS EC50 (nM) |
|---|---|
| EP381 | 0.766 |
| EP382 | 3.814 |
| EP383 | 0.846 |
| EP384 | 1.362 |

(b) Binding Activity to CD3+ Cells

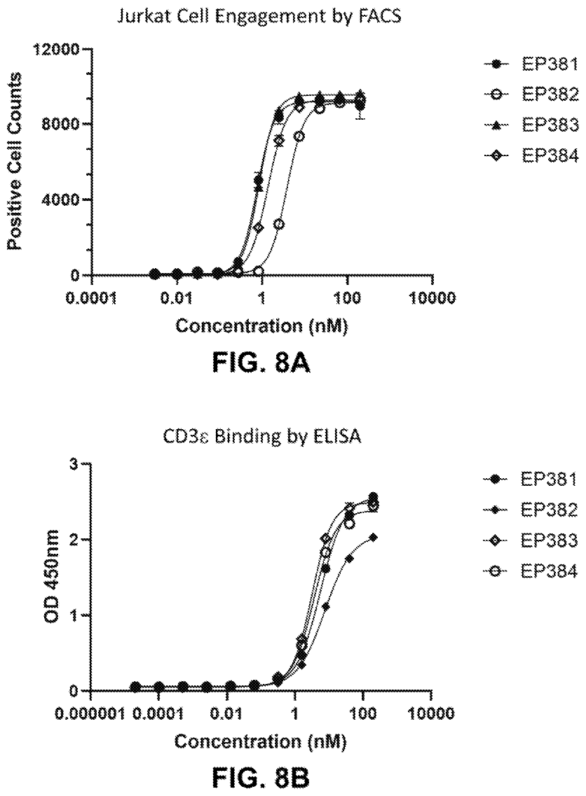

A similarly FACS assay was performed to examine the binding activity of the exemplary bispecific anti-CD19/anti-CD3 antibodies to CD3+ Jurkat T cells, following the descriptions above. As shown in FIG. 8A, all of the bispecific antibodies as indicated were capable of binding to CD3⁺ Jurkat cells.

Further, an ELISA assay was developed to determine the EC50 of anti-CD19/CD3 BiTE antibodies. Briefly, 384 well plate was immobilized with human CD3ε/Fc at final concentration of 2 μg/mL in 1×PBS in total volume of 25 uL per well. The plate was incubated overnight at 4° C. followed by blocking with 80 uL of superblock per well for 1 hour. Anti-CD19/CD3 BiTE proteins were serial diluted, and 25 μL was added to CD3ε immobilized wells and incubated for 1 hour with shaking. The CD3ε binding was detected by adding 25 μL of anti-Flag HRP diluted at 1:5000 in 1×PBST. In between each step, the plate was washed 3 times with 1×PBST in a plate washer. The plate was then developed with 20 μL of TMB substrate for 5 mins and stopped by adding 20 μL of 2N sulfuric acid. The plate was read at OD450 nm Biotek plate reader and then plotted in Prism 8.1 software. As shown in FIG. 8B, all of the BiTE antibodies tested in this assay showed binding activity to CD3ζ. EC50 was calculated as shown in Table 7 below.

TABLE 7
EC50 Values of BiTE Antibodies Measured by ELISA

| | ELISA EC50 (nM) |
|---|---|
| EP381 | 5.57 |
| EP382 | 7.585 |
| EP383 | 3.171 |
| EP384 | 3.641 |

### Example 9: Anti-CD19/Anti-CD3 BiTE Antibody CTL Assay with Primary T Cells

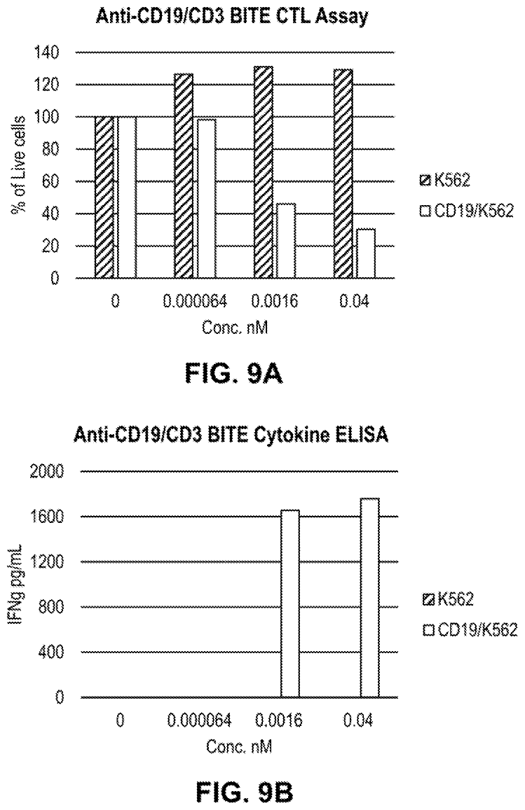

K562 and CD19/K562 GFP tagged target cells were plated at 20,000 cells per well onto 96 well black plate in 50 μL media. Anti-CD19/CD3 BiTE antibodies and controls were 5-fold serial diluted in 50 μL of media. The BiTE antibodies were then incubated with target cells for 1 hour at room temperature. 100,000 T cells in 50 μL were added to target cells that pre-incubated with BiTE antibody in each well at effector to target cell ratio of 5:1. Assay plate was incubated at 37 C for 48 hrs and imaged every 2 hours by Cytation 5 instrument. After 48 hrs, the supernatant was harvested for IFN-gamma ELISA assay. GFP tagged live target cells were counted by flow cytometry. As shown in FIG. 9A, the BiTE antibody showed cytotoxicity activity against CD19⁺ K562 cells, but not CD19⁻ K562 cells, using EP381 as an example.

### Example 10: Anti-CD19/Anti-CD3 BiTE Antibody IFNg Assay with Primary T Cells

IFN-gamma was detected with Human IFN-gamma Duoset ELISA kit (R&D System) post CTL assay. Briefly, supernatant was collected after CTL assay terminated. Recombinant IFN-gamma was serial diluted and included in the assay to create standard curve. Supernatant IFNg and recombinant IFNg were assayed following the manufacture's protocol provided. The data was analyzed using Prism 8.0 software. Consistent with the CTL assay results discussed in Example 9 above, the BiTE antibody induced IFN-gamma secretion when incubated with CD19⁺ K562 cells, but not with CD19⁻ K562 cells, using EP381 as an example. FIG. 9B.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An isolated antibody that binds CD19, wherein the antibody binds to the same epitope as a reference antibody or competes against the reference antibody from binding to CD19, and wherein the reference antibody is selected from the group consisting of EP142-D9, EP187-A12, EP188-A01, and EP188-B10.

2. The isolated antibody of claim 1, wherein the antibody comprises:
(a) a heavy chain complementary determining region 1 (HC CDR1), a heavy chain complementary determining region 2 (HC CDR2), and a heavy chain complementary determining region 3 (HC CDR3), wherein the HC CDR1, HC CDR2, and HC CDR3 collectively are at least 80% identical to the heavy chain CDRs of the reference antibody; and/or
(b) a light chain complementary determining region 1 (LC CDR1), a light chain complementary determining region 2 (LC CDR2), and a light chain complementary determining region 3 (LC CDR3), wherein the LC CDR1, LC CDR2, and LC CDR3 collectively are at least 80% identical to the light chain CDRs of the reference antibody.

3. The isolated antibody of claim 1 or claim 2, wherein the HC CDRs of the antibody collectively contain no more than 8 amino acid residue variations as compared with the HC CDRs of the reference antibody; and/or wherein the LC CDRs of the antibody collectively contain no more than 8 amino acid residue variations as compared with the LC CDRs of the reference antibody.

4. The isolated antibody of any one of claims 1-3, wherein the antibody comprises a V_{H} that is at least 85% identical to the V_{H} of the reference antibody, and/or a V_{L} that is at least 85% identical to the V_{L} of the reference antibody.

5. The isolated antibody of any one of claims 1-4, wherein the antibody has a binding affinity of less than 10 nM to CD19 expressed on cell surface.

6. The isolated antibody of claim 5, wherein the antibody has a binding affinity of less than 1 nM to CD19 expressed on cell surface.

7. The isolated antibody of claim 1, which comprises the same heavy chain complementary determining regions (HC CDRs) and the same light chain complementary determining regions (LC CDRs) as the reference antibody.

8. The isolated antibody of claim 7, which comprises the same V_{H} and the same V_{L} as the reference antibody.

9. The isolated antibody of any one of claims 1-8, wherein the antibody is a human antibody or a humanized antibody.

10. The isolated antibody of any one of claims 1-9, wherein the antibody is a full-length antibody or an antigen-binding fragment thereof.

11. The isolated antibody of any one of claims 1-9, wherein the antibody is a single-chain antibody (scFv).

12. The isolated antibody of claim 11, wherein the antibody comprises an amino acid sequence selected the group consisting of SEQ ID NOs:11-14.

13. The isolated antibody of any one of claims 1-11, which is a bispecific antibody that binds CD19 and a second antigen.

14. The isolated antibody of claim 13, wherein the second antigen is CD3.

15. The isolated antibody of claim 14, wherein the antibody comprises a first scFv that binds CD19 and a second scFv that binds CD3.

16. The isolated antibody of claim 15, wherein the first scFv is set forth in claim 11 or claim 12.

17. The isolated antibody of claim 15 or claim 16, wherein the second scFv comprises a heavy chain variable domain comprising the amino acid sequence of SEQ ID NO: 42 and a light chain variable domain comprising the amino acid sequence of SEQ ID NO: 43.

18. The isolated antibody of claim 15, which comprises the amino acid sequence of any one of SEQ ID NOs: 40, 45, 47, and 49.

19. A nucleic acid or a set of nucleic acids, which collectively encodes the antibody of any one of claims 1-18.

20. The nucleic acid or the set of nucleic acids of claim 19, which is a vector or a set of vectors.

21. The nucleic acid or the set of nucleic acids or claim 20, wherein the vector is an expression vector.

22. A host cell comprising the nucleic acid or the set of nucleic acids of any one of claims 19-21.

23. A pharmaceutical composition comprising the antibody of any one of claims 1-18, the nucleic acid or nucleic acids of any one of claims 19-21, or the host cell of claim 22, and a pharmaceutically acceptable carrier.

24. A method for inhibiting CD19 in a subject, comprising administering to a subject in need thereof any effective amount of the pharmaceutical composition of claim 23.

25. The method of claim 24, wherein the subject is a human patient having CD19⁺ pathogenic cells.

26. The method of claim 24 or claim 25, wherein the subject is a human patient having cancer.

27. The method of claim 26, wherein the human patient has CD19⁺ cancer cells.

28. A method for detecting presence of CD19, comprising:
(i) contacting an antibody of any one of claims 1-18 with a sample suspected of containing CD19, and
(ii) detecting binding of the antibody to CD19.

29. The method of claim 28, wherein the antibody is conjugated to a detectable label.

30. The method of claim 28 or claim 29, wherein the CD19 is expressed on cell surface.

31. The method of any one of claims 28-30, wherein the contacting step is performed by administering the antibody to a subject.

32. A method of producing an antibody binding to CD19, comprising:
(i) culturing the host cell of claim 22 under conditions allowing for expression of the antibody that binds CD19; and
(ii) harvesting the antibody thus produced from the cell culture.


SEQUENCE LISTING (49 sequences)

SEQ ID NO: 1 - protein, 120 aa, Mouse
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 107 aa, Mouse
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 125 aa, Human
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSS

SEQ ID NO: 4 - protein, 106 aa, Human
QPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVL

SEQ ID NO: 5 - protein, 121 aa, Human
QVQLVQSGGGVVQPGKSLRLSCAASGFPFSSYTMHWVRQPPGEGLEWVALISYDGRNLYYADSVKGRFTISRDNSYNSLYLQLSGLRAEDTALYYCARDINRDHFYGMDLWGPGTTVTVSS

SEQ ID NO: 6 - protein, 108 aa, Human
SYELTQPPSVSVAPGQTARIPCGGTNIGSKGVHWYQQKPGQAPVLVIYYDHSRPSGIPERFSGSNSGNTAALTISRVEAGDEADYYCQVWEGTSDHPVFGGGTKLTVL

SEQ ID NO: 7 - protein, 118 aa, Human
EVQLVESGAEVKKPGASVKVSCKASGYTFTSYYMHWVRQAPGQGLEWMGIINPSGGSTSYAQKFQGRVTITADESTSTAYMELSSLRSEDTAVYYCAREGGYKDFDYWGQGTLVTVSS

SEQ ID NO: 8 - protein, 107 aa, Human
DIVMTQSPSSLSASVGDRVTVTCRASQSIDTYLNWYQQKPGKAPKLLIYTASTLQSGVPSRFSGSGSGTDFTLTISSLQPEDFATYYCQQSYSAPRTFGQGTKVEIK

SEQ ID NO: 9 - protein, 126 aa, Human
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREALPWDKWYGGYEAFDYWGQGTLVTVSS

SEQ ID NO: 10 - protein, 107 aa, Human
NIQMTQSPSSLSASIGDRVTITCRASQGLNTYVAWYQQKPGKAPKLLMYDASTLQSGVPARFSGTGSGTDFTLTISSLQPEDFATYYCQQVNSFGYTFGQGTKLEIK

SEQ ID NO: 11 - protein, 246 aa, Unknown
  ScFv
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVL

SEQ ID NO: 12 - protein, 244 aa, Unknown
  ScFv
QVQLVQSGGGVVQPGKSLRLSCAASGFPFSSYTMHWVRQPPGEGLEWVALISYDGRNLYYADSVKGRFTISRDNSYNSLYLQLSGLRAEDTALYYCARDINRDHFYGMDLWGPGTTVTVSSGGGGSGGGGSGGGGSSYELTQPPSVSVAPGQTARIPCGGTNIGSKGVHWYQQKPGQAPVLVIYYDHSRPSGIPERFSGSNSGNTAALTISRVEAGDEADYYCQVWEGTSDHPVFGGGTKLTVL

SEQ ID NO: 13 - protein, 240 aa, Unknown
  ScFv
EVQLVESGAEVKKPGASVKVSCKASGYTFTSYYMHWVRQAPGQGLEWMGIINPSGGSTSYAQKFQGRVTITADESTSTAYMELSSLRSEDTAVYYCAREGGYKDFDYWGQGTLVTVSSGGGGSGGGGSGGGGSDIVMTQSPSSLSASVGDRVTVTCRASQSIDTYLNWYQQKPGKAPKLLIYTASTLQSGVPSRFSGSGSGTDFTLTISSLQPEDFATYYCQQSYSAPRTFGQGTKVEIK

SEQ ID NO: 14 - protein, 248 aa, Unknown
  ScFv
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREALPWDKWYGGYEAFDYWGQGTLVTVSSGGGGSGGGGSGGGGSNIQMTQSPSSLSASIGDRVTITCRASQGLNTYVAWYQQKPGKAPKLLMYDASTLQSGVPARFSGTGSGTDFTLTISSLQPEDFATYYCQQVNSFGYTFGQGTKLEIK

SEQ ID NO: 15 - protein, 5 aa, Human
GYYWT

SEQ ID NO: 16 - protein, 16 aa, Human
EINHGGSSNYNPSLKS

SEQ ID NO: 17 - protein, 17 aa, Human
GLGYRSGWYEVENAFDI

SEQ ID NO: 18 - protein, 11 aa, Human
GGNKIESRSVH

SEQ ID NO: 19 - protein, 7 aa, Human
DDGARPS

SEQ ID NO: 20 - protein, 9 aa, Human
QVWDGSSVI

SEQ ID NO: 21 - protein, 5 aa, Human
SYTMH

SEQ ID NO: 22 - protein, 17 aa, Human
LISYDGRNLYYADSVKG

SEQ ID NO: 23 - protein, 12 aa, Human
DINRDHFYGMDL

SEQ ID NO: 24 - protein, 11 aa, Human
GGTNIGSKGVH

SEQ ID NO: 25 - protein, 7 aa, Human
YDHSRPS

SEQ ID NO: 26 - protein, 11 aa, Human
QVWEGTSDHPV

SEQ ID NO: 27 - protein, 5 aa, Human
SYYMH

SEQ ID NO: 28 - protein, 17 aa, Human
IINPSGGSTSYAQKFQG

SEQ ID NO: 29 - protein, 9 aa, Human
EGGYKDFDY

SEQ ID NO: 30 - protein, 11 aa, Human
RASQSIDTYLN

SEQ ID NO: 31 - protein, 7 aa, Human
TASTLQS

SEQ ID NO: 32 - protein, 9 aa, Human
QQSYSAPRT

SEQ ID NO: 33 - protein, 5 aa, Human
GYYMH

SEQ ID NO: 34 - protein, 17 aa, Human
WINPNSGGTNYAQKFQG

SEQ ID NO: 35 - protein, 17 aa, Human
EALPWDKWYGGYEAFDY

SEQ ID NO: 36 - protein, 11 aa, Human
RASQGLNTYVA

SEQ ID NO: 37 - protein, 7 aa, Human
DASTLQS

SEQ ID NO: 38 - protein, 9 aa, Human
QQVNSFGYT

SEQ ID NO: 39 - protein, 531 aa, Unknown
  Bispecific antibody
METDTLLLWVLLLWVPGSTGQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLHHHHHH

SEQ ID NO: 40 - protein, 505 aa, Unknown
  Bispecific antibody
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 41 - protein, 20 aa, Unknown
  Signal peptide
METDTLLLWVLLLWVPGSTG

SEQ ID NO: 42 - protein, 123 aa, Unknown
  VH of anti-CD3 antibody
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTV

SEQ ID NO: 43 - protein, 109 aa, Unknown
  VL of anti-CD3 antibody
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 44 - protein, 529 aa, Unknown
  Bispecific antibody
METDTLLLWVLLLWVPGSTGQVQLVQSGGGVVQPGKSLRLSCAASGFPFSSYTMHWVRQPPGEGLEWVALISYDGRNLYYADSVKGRFTISRDNSYNSLYLQLSGLRAEDTALYYCARDINRDHFYGMDLWGPGTTVTVSSGGGGSGGGGSGGGGSSYELTQPPSVSVAPGQTARIPCGGTNIGSKGVHWYQQKPGQAPVLVIYYDHSRPSGIPERFSGSNSGNTAALTISRVEAGDEADYYCQVWEGTSDHPVFGGGTKLTVLGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLHHHHHH

SEQ ID NO: 45 - protein, 503 aa, Unknown
  Bispecific antibody
QVQLVQSGGGVVQPGKSLRLSCAASGFPFSSYTMHWVRQPPGEGLEWVALISYDGRNLYYADSVKGRFTISRDNSYNSLYLQLSGLRAEDTALYYCARDINRDHFYGMDLWGPGTTVTVSSGGGGSGGGGSGGGGSSYELTQPPSVSVAPGQTARIPCGGTNIGSKGVHWYQQKPGQAPVLVIYYDHSRPSGIPERFSGSNSGNTAALTISRVEAGDEADYYCQVWEGTSDHPVFGGGTKLTVLGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 46 - protein, 525 aa, Unknown
  Bispecific antibody
METDTLLLWVLLLWVPGSTGEVQLVESGAEVKKPGASVKVSCKASGYTFTSYYMHWVRQAPGQGLEWMGIINPSGGSTSYAQKFQGRVTITADESTSTAYMELSSLRSEDTAVYYCAREGGYKDFDYWGQGTLVTVSSGGGGSGGGGSGGGGSDIVMTQSPSSLSASVGDRVTVTCRASQSIDTYLNWYQQKPGKAPKLLIYTASTLQSGVPSRFSGSGSGTDFTLTISSLQPEDFATYYCQQSYSAPRTFGQGTKVEIKGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLHHHHHH

SEQ ID NO: 47 - protein, 499 aa, Unknown
  Bispecific antibody
EVQLVESGAEVKKPGASVKVSCKASGYTFTSYYMHWVRQAPGQGLEWMGIINPSGGSTSYAQKFQGRVTITADESTSTAYMELSSLRSEDTAVYYCAREGGYKDFDYWGQGTLVTVSSGGGGSGGGGSGGGGSDIVMTQSPSSLSASVGDRVTVTCRASQSIDTYLNWYQQKPGKAPKLLIYTASTLQSGVPSRFSGSGSGTDFTLTISSLQPEDFATYYCQQSYSAPRTFGQGTKVEIKGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 48 - protein, 533 aa, Unknown
  Bispecific antibody
METDTLLLWVLLLWVPGSTGQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREALPWDKWYGGYEAFDYWGQGTLVTVSSGGGGSGGGGSGGGGSNIQMTQSPSSLSASIGDRVTITCRASQGLNTYVAWYQQKPGKAPKLLMYDASTLQSGVPARFSGTGSGTDFTLTISSLQPEDFATYYCQQVNSFGYTFGQGTKLEIKGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLHHHHHH

SEQ ID NO: 49 - protein, 507 aa, Unknown
  Bispecific antibody
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREALPWDKWYGGYEAFDYWGQGTLVTVSSGGGGSGGGGSGGGGSNIQMTQSPSSLSASIGDRVTITCRASQGLNTYVAWYQQKPGKAPKLLMYDASTLQSGVPARFSGTGSGTDFTLTISSLQPEDFATYYCQQVNSFGYTFGQGTKLEIKGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL